(19) United States
(12) Patent Application Publication
(10) Pub. No.: US 2024/0047060 A1
(43) Pub. Date: Feb. 8, 2024

(54) Handheld Medical Robot Workstation and Application Method Therefor, and System

(71) Applicant: Xiamen Brana Design Co., Ltd., Xiamen, Fujian Province (CN)

(72) Inventor: Zhongtang WANG, Xiamen (CN)

(21) Appl. No.: 18/031,007

(22) PCT Filed: Feb. 26, 2021
(86) PCT No.: PCT/CN2021/078119

§ 371 Date: Oct. 16, 2023

(30) Foreign Application Priority Data
Feb. 26, 2021 (WO) PCT/CN2021/078119

(51) Int. Cl. G16H 40/67; A61B 5/151; A61B 5/00; G01N 35/00; G16H 20/60

(52) U.S. Cl. CPC G16H 40/67; A61B 5/15101; A61B 5/7475; A61B 5/7465; G01N 35/00029; G16H 20/60; G01N 2035/00108

(57) ABSTRACT

A handheld medical robot workstation includes a test strip detection device, a medical robot body, a blood test pen device, and an inspection handpiece. A user can deploy the handheld medical robot workstation in a modular manner. Through human-machine interaction, a family member can create health files, obtain health management and diagnosis and treatment services by her/himself, and a family doctor can track the health data of the family member in real time. In case of abnormal health, medical and drug delivery services can be provided in a timely manner

### CROSS REFERENCE OF RELATED APPLICATION

This application is a U.S. National Stage Entry Under 35 U.S.C. 371 of international application No. PCT/CN2021/078119, filed Feb. 26, 2021, which claims the benefit of priority under 35 U.S.C. § 119 to a Chinese application, application number 202011101610.2, filed on Oct. 9, 2020, which are incorporated herewith by reference in their entirety.

### BACKGROUND OF THE PRESENT INVENTION

### Field of Invention

The present invention relates to a field of medical service robots, more particularly to a handheld medical robot workstation and system and usage thereof.

### Description of Related Arts

The world today is far from perfect. The distribution of medical resources is uneven. A few people enjoy more high-quality medical resources. Most people suffer from diseases due to lack of medical care and medicine. The Internet is far from universal. Half of the world's people cannot enjoy the convenience brought by Internet technology.

The efforts made by the U.S. and Chinese technology giant to achieve global network connectivity and popularize smart phones have brought hope for the eradication of poverty and the fight against disease.

Especially in some economically backward countries and remote villages in some countries, there are a large number of groups whose medical and health needs cannot be fully met. How to provide medical and health services for this group is a worldwide problem.

In addition, even in countries or regions with relatively developed medical conditions, it is not easy for family members to make an appointment for high-quality medical services when they are ill. Often, they have to go to the hospital to see the emergency only when the disease has progressed to a critical stage, which not only delays the disease, and aggravates the pain, but also sacrifices work and rest time.

Therefore, we have designed a medical robot workstation, which is small in size, and can be held by adults with one hand. It is flexible to select some intelligent diagnostic and therapeutic components that are combined by plugging or building blocks. A family member can complete a health examination, health management, and basic diagnosis and treatment by her/himself, which will improve the unbalanced distribution of medical resources and meet the medical and health needs of economically backward countries and remote villages, reduce a waste of social and medical resources and promote human health.

### SUMMARY OF THE PRESENT INVENTION

An objective of the invention is to provide a handheld medical robot workstation and application method and system thereof. A user can complete health examination, health management, and basic diagnosis and treatment operations at home, so as to meet medical and health needs, reduce waste of medical and social resources, and promote human health.

A handheld medical robot workstation according to an embodiment of the present invention comprises a medical robot body, a test strip detection device, a plurality of inspection handpieces, a blood detection pen device, a component storage box, and reagent consumables. In a rest state, the test strip detection device, the inspection handpieces, the blood detection pen device, and the reagent consumables can be stored in the component storage box. In a working state, the test strip detection device, the inspection handpieces, the blood detection pen device can be connected with the medical robot body.

The medical robot body according to the embodiment of the present invention comprises a power module, a wireless communication module, a data storage and processing module, a display module, a plug slot module, an environmental monitoring module, an operation button module, a biometric module, a human-machine interaction module, and a data security module. A back side of the medical robot body is provided with a bracket so that it can stand obliquely on a desktop, and a bottom end of the medical robot body is provided with another bracket, which makes it stand upright on a ground.

The test strip detection device according to the embodiment of the present invention comprises a groove part and a detection part, a bottom of the groove part is connected with the detection part through a bayonet, an upper part of the detection part is connected with the groove part through a bayonet, a lower part of the detection part is connected with the medical robot body through a bayonet and a plug, the plug is equipped with a power supply and a data line interface, the detection part comprises a detection head and a lead screw system, the detection head and the lead screw system connect the power supply and data line interface, and the detection head is fixed on the slider of the lead screw system.

The inspection handpiece according to the embodiment of the present invention comprises a vital sign monitoring apparatus, an intelligent stethoscope, an intelligent pulse detector, a computer vision sensor probe, an intelligent ultrasonic probe, an intelligent electrocardiogram (ECG) electrode lead probe, an intelligent electroencephalogram (EEG) electrode lead probe, an intelligent respiratory function inspection probe, an expiratory component detection sensor, an intelligent capsule gastrointestinal endoscope, and an intelligent endoscope.

The blood detection pen device according to the embodiment of the present invention comprises a housing, a puncture component, a blood collection component, a blood cell counting and classifying component, a blood electrolyte detecting component, and a waste liquid collecting sac. The blood collection component includes a capillary tube, an anticoagulant coating, and an intelligent micro pump. A plurality of branches of the capillary tube enters the blood cell counting and classifying component and the blood electrolyte detecting component respectively. After the blood flows out of the blood cell counting and sorting unit and the blood electrolyte detection component, it can be driven by the intelligent micro pump and enter the waste liquid collection sac.

The component storage box according to the embodiment of the present invention comprises a power adapter, a power socket, a power cord, a battery, and a plurality of storage compartments, the storage compartments are provided with circuit interfaces, a size and shape of the storage compartment can be adapted to a collected component, and the collected component includes but is not limited to the groove part, the detection part, the inspection handpieces, and the blood detection pen device.

The reagent consumables of according to the embodiment of the present invention comprises a test strip, a buffer, a cleaning detergent, a triangular needle, a capillary tube and a waste liquid collection sac.

Preferably, the plug slot module comprises at least one plug slot, which includes a circuit interface, a data interface and a fixed bayonet.

Preferably, the inspection handpiece adopts a wireless technology scheme, which includes a power supply, a wireless communication equipment, a signal transmitting equipment, and a signal acquisition equipment. A data can be transmitted to the data storage and processing module of the medical robot body through wireless communication.

A method of a handheld medical robot workstation according to the embodiment of the present invention comprises a plurality of steps as follows:
110: a user submits an application for acquiring the handheld medical robot workstation;
120: a remote expert service provides simulation training; 130: the user deploys the handheld medical robot workstation;
140: the user implement a daily health management, including a home environment monitoring, a diet health monitoring, a weight management, a vital signs monitoring;
150: the user conducts physical examination at home;
160: the user seeks medical advice; and
170: a family members' health records can be built.

Preferably, the user deployment process 200 of the handheld medical robot workstation according to the embodiment of the present invention includes a plurality of steps as follows:
210: determining a requirement;
220: configuring a device combination and service scheme of the handheld medical robot workstation;
230: assembling functional modulars in a pluggable building block manner; and
240: creating medical and health data files for family members with human-machine interaction.

Preferably, the vital signs monitoring process 300 of the handheld medical robot workstation of an embodiment of the present invention includes a plurality of steps as follow:
310: configuring the vital signs monitoring equipment in the handheld medical robotic workstation;
320: selecting the vital signs monitoring interface, removing the intelligent detection sensor probe, initiating monitoring, and acquire monitoring data; and
330: setting a value range of family members, if out of the value range, showing a warning, and sending the monitoring data to a family doctor synchronously.

Preferably, the physical examination in home process 400 of the handheld medical robot workstation of an embodiment of the present invention includes a plurality of steps as follows:
410: planning a physical examination of a family member according to a family health plan;
420: setting up individualized physical exam items for the family member referring to family physician recommendation;
430: renting the inspection handpieces, the blood detection pen device, and purchasing the reagent consumables;
440: remotely guided by family physician, self-help implementing physical exam at home;
450: physical examination data being encrypted and uploaded to a cloud server to create a family member health file; and
460: family physician obtaining the physical examination data so as to give medical health advice.

Preferably, the seeking medical advice process 500 of the handheld medical robot workstation of an embodiment of the present invention includes a plurality of steps as follows:
510: if feeling unwell, a family member being able to appoint with a family physician;
520: through a human-machine interaction and remote questioning, monitoring vital signs, cardiopulmonary auscultation, traditional Chinese medicine seeing and physical examination by self-help or assistance from family members;
530: implementing ancillary tests, including excreta and/or blood detection, handpieces inspection;
540: definite diagnosis;
550: developing a protocol for diagnosis and treatment;
560: treatment; and
570: follow up.

A system of a handheld medical robotic workstation according to the embodiment of the present invention comprises a medical AI, an Internet of things system, a supply chain system, a logistics system, a cloud service, an operating system, and an application software which perform an instruction issued by a data storage and processing module and assist in the completion of operation and method process of the handheld medical robotic workstation.

(1) The handheld medical robotic workstation includes hardware and software, with a small size of the medical robot body as a smartphone or a smart plate for host computer, with a play screen for human-machine interaction and shared monitor, with an upper end to set up a test strip detection device, with both sides and lower end to set up the inspection handpieces, the blood detection pen device, and the environmental monitoring device, with the component storage box further as a charging device.

(2) The test strip detection device can be configured to daily dietary safety testing and dry chemical dipstick test cards for blood and excretory components. The inspection handpieces include equipment for monitoring vital signs, auscultators, pulse image detectors, visual sensors, ultrasound probes, electrocardiography probes, electroencephalography probes, respiratory examination probes, expiratory component sensors, endoscopy, being able to meet most of the daily health and clinical diagnosis and treatment needs of physics imaging examination. The volume of peripheral blood obtained by the blood detection pen device can meet most of the blood examination needs, and the integrated blood examination equipment and blood composition analysis equipment use flow cytometry technology and electrochemical sensor array technology, being able to replace routine large examination equipment, and meet the low-frequency clinical diagnosis and treatment needs of a user.

(3) When a user deploys a handheld medical robotic workstation, one or more of components can be selectively configured according to demand, such as the test strip detection device, inspection handpieces, blood detection pen device, environmental monitoring device, and other components or reagent consumables with low frequency can be purchased when required or rented. Moreover, the handheld medical robotic workstation does not need to be purchased, a user can charter out to manufacturers or service manufacturers to further save financial burden on the user and increase an efficiency of utilization of the handheld medical robotic workstation.

(4) Because of the handheld medical robot workstation and the accompanying components with a small shape, an easy logistics transport and deployment, use simple and versatile, it will help to make it to thousands of households, meet the needs of economically backward countries, remote areas, ordinary households and have easy access to medical health services, thereby partial replacement of complex expensive medical robots and scarce quality medical services.

### BRIEF DESCRIPTION OF THE DRAWINGS

In order to more clearly illustrate the technical schemes in the embodiments or prior art, the embodiments required in the description of the embodiments or prior art are introduced below in brief, and it is obvious that, for those of ordinary skill in the art, other drawings can also be obtained according to such embodiments without exerting inventive labor. Furthermore, these presentations do not constitute a qualification of the examples.

FIG. 1 is a schematic structure of a medical robot body according to a preferred embodiment of the present invention.

FIG. 2 is a schematic structure of a handheld medical robotic workstation with a dorsal stent support according to a preferred embodiment of the present invention.

FIG. 3 is a schematic structure of a handheld medical robotic workstation with a bottom end tripod support according to a preferred embodiment of the present invention.

FIG. 4A is a schematic structure of a groove part of a test strip detection device according to a preferred embodiment of the present invention.

FIG. 4B is a cross-sectional schematic structure of a groove part of a test strip detection device according to a preferred embodiment of the present invention.

FIG. 4C is a schematic structure of a groove part of a test strip detection device with a test strip according to a preferred embodiment of the present invention.

FIG. 5A is an outer schematic structure of a detection part of a test strip detection device according to a preferred embodiment of the present invention.

FIG. 5B is an internal schematic structure of a detection part of a test strip detection device according to a preferred embodiment of the present invention.

FIG. 6 is a schematic structure of a handheld medical robotic workstation with a plurality of components according to a preferred embodiment of the present invention.

FIG. 7 is a schematic structure of a blood detection pen device including a blood sampling component and a detection component according to a preferred embodiment of the present invention.

FIG. 8 is a schematic structure of a piercing part of a blood detection pen device according to a preferred embodiment of the present invention.

FIG. 9 is a schematic structure of a housing of a blood detection pen device according to a preferred embodiment of the present invention.

FIG. 10A is an exterior schematic structure of a component storage box according to a preferred embodiment of the present invention.

FIG. 10B is a coronal section schematic structure of a component storage box according to a preferred embodiment of the present invention.

### DETAILED DESCRIPTION OF THE PREFERRED EMBODIMENT

A handheld medical robotic workstation of a preferred embodiment of the present invention comprises a medical robot body, a test strip detection device, a plurality of inspection handpieces, a blood detection pen device, a component storage box, and a reagent consumables, the test strip detection device, the inspection handpieces, the blood detection pen device and reagent consumables can be taken in the component storage box when in non-working state, and the test strip detection device, the inspection handpieces, and the blood detection pen device can be connected to the medical robotic body in a working state.

The medical robot body comprises a power module, a wireless communication module, a data storage and processing module, a display module, a pluggable slot module, an environmental monitoring module, an operational button module, a biometric module, a human-machine interaction module, and a data security module, the medical robot body can be set a dorsally racket for inclining to a table top, and a bottom racket for standing upright to the ground.

The test strip detection device comprises a groove part and a detection part, the detection part includes a detection head and a lead screw system, and the detection head is fixed on the slider of the lead screw system.

The inspection handpiece comprises a vital sign monitoring device, an intelligent stethoscope, an intelligent pulse detector, a computer vision sensor probe, an intelligent ultrasonic probe, an intelligent ECG electrode lead probe, an intelligent EEG electrode lead probe, an intelligent respiratory function inspection probe, an expiratory component detection sensor, an intelligent capsule gastrointestinal endoscope, and an intelligent endoscope.

The blood detection pen device comprises a housing, a puncture component, a blood collection component, a blood cell counting and classifying component, a blood electrolyte detecting component, and a waste liquid collecting sac.

The component storage box comprises a power adapter, a power socket, a power cord, a battery, and a plurality of storage compartments, and the collected component includes but is not limited to the groove part, the detection part, the inspection handpieces, and the blood detection pen device.

A method of a handheld medical robot workstation of an better embodiment of the present invention comprises a plurality of steps as follows:
110: a user submits an application for acquiring the handheld medical robot workstation;
120: a remote expert service provides simulation training;
130: the user deploys the handheld medical robot workstation;
140: the user implement a daily health management;
150: the user conducts physical examination at home;
160: the user seeks medical advice; and
170: a family members' health records can be built.

A system of a handheld medical robotic workstation of a better embodiment of the present invention comprises a medical AI, an Internet of things system, a supply chain system, a logistics system, a cloud service, an operating system, and an application software configured to complete operation and method process of the handheld medical robotic workstation.

To achieve the objectives, technical schemes, and advantages of the present invention clearer, the invention is further elucidated in conjunction with specific examples below. It is to be understood that these examples serve only to illustrate the invention and not to limit the scope of the invention, and after reading the invention, modifications of the various equivalent forms of the invention by persons skilled in the art fall within the scope defined by the claims appended to the application.

It needs to be stated that the superior, inferior, left, right, anterior, posterior, dorsal, lateral, first, second, etc. orientations of the present examples, which are only mutually relative concepts or are referenced to the normal state of use of the product, should not be considered restrictive.

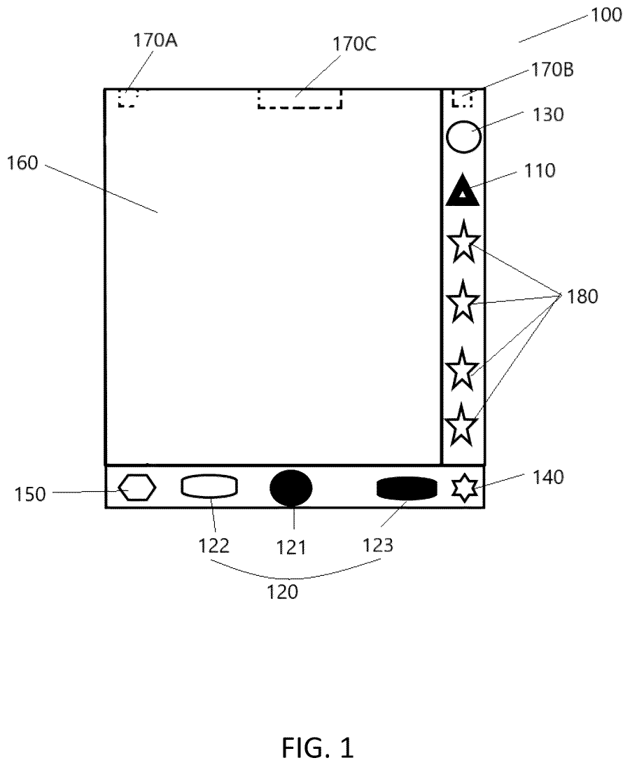

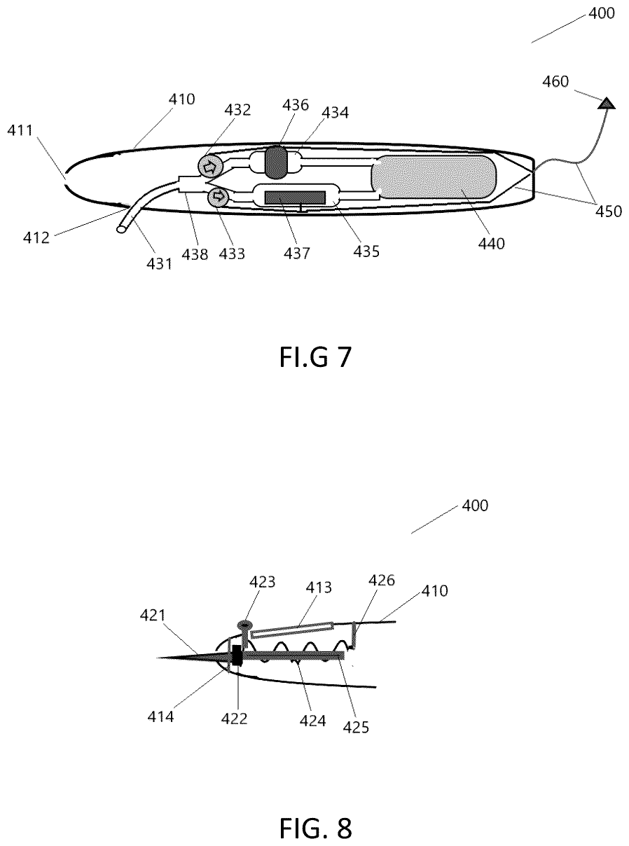

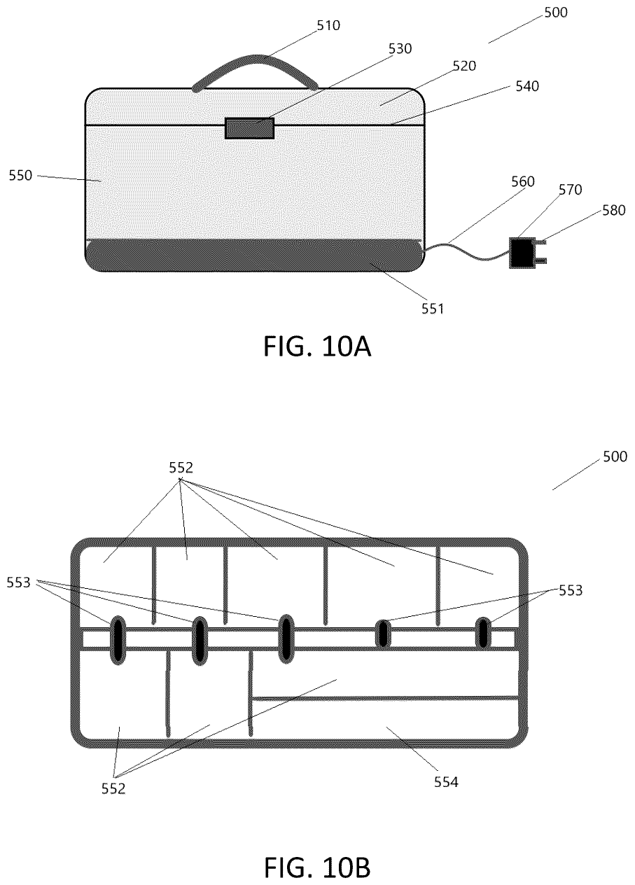

Referring to FIGS. 1 to 10B, the preferred embodiment of the present invention reveals a handheld medical robotic workstation, an application method, and an application system. The handheld medical robotic workstation of an embodiment of the present invention can comprise a medical robot body 100, a test strip detection device 200, a plurality of inspection handpieces 300, a blood detection pen device 400, an environmental monitoring module, a dietary health monitoring module, a weight/exercise management module, a component storage box 500, and reagent consumables 600.

As shown in FIG. 1, according to one embodiment of the present invention a medical robot body 100 of a hand-held medical robot workstation can comprise a power module, a wireless communication module, a data storage and processing module, a display module, a pluggable slot module, a biometric module, a human-machine interaction module, a data security module, an environmental monitoring device 110, an operation button 120, a camera 130, a microphone 140, and a sound player 150.

The display module of one embodiment of the present invention can include a touchscreen display 160, which can routinely display a power and a state of charge of the power module, a network signal intensity, a functional status of a probing part 220, an environmental monitoring device 110, an inspection handpiece 300, a blood detection pen device 400, and a human-machine interaction interface.

The pluggable slot module of an embodiment of the present invention can be set to a periphery of the medical robot body 100, which can include a first left pluggable port 170A, a first right pluggable port 170B, a plug 170C, and a plurality of pluggable interfaces 180, the first left pluggable port 170A, a first right pluggable port 170B and the plug 170C can be at an upper end of the medical robot body 100 configured to connect the probing part 220, and pluggable interfaces 180 can include a circuit interface, a data interface and a fixed card slot configured to accept the probing part 220 and the inspection handpieces 300. To further reduce the size of the medical robot body 100 and obtain a concise appearance, the pluggable interface 180 can be set to a side or dorsal side of the medical robot body 100.

The operation button 120 of an embodiment of the present invention can include a power key 121, a detect key 122, a sound adjust key 122, and a display screen luminance adjust key 123. The power key 121 and the detect key 122 can be shared. In addition, the power key 121, the detect key, the sound adjust key 122 and the display screen luminance adjust key 123 can be set to a side of the medical robot body 100, and the detect key 122, the sound adjust key 122 and the display screen luminance adjust key 123 can be set to inside the touchscreen display 160 as a virtual key, to reduce the size and simplify the medical robot body 100, thus obtaining a more humane human-machine interaction and reducing the cost of fabrication.

The environmental monitoring device 110 of an embodiment of the present invention can include a sensor array which can detect including, but not limited to, temperature, humidity, CO, CO2, particulate matter (PM) 2.5, PM10, NH3, formaldehyde, and gamma rays. Of course, the sensor array can be appropriately increased or decreased depending on a user requirement.

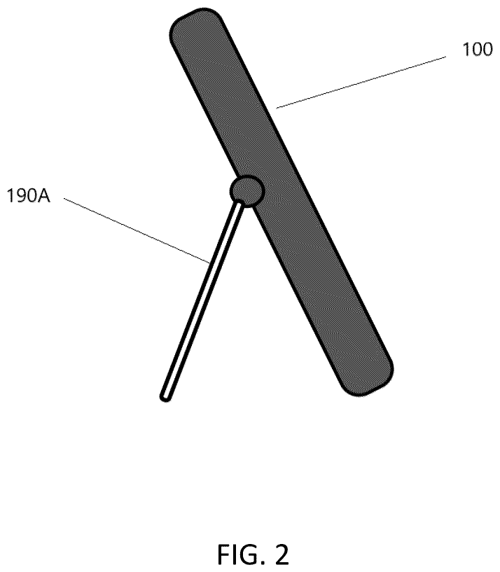

As shown in FIG. 2, a medical robot body 100 of an embodiment of the present invention can includes a dorsal bracket 190A on a back side configured to lean the medical robot body 100 on a table surface and liberate a user hand.

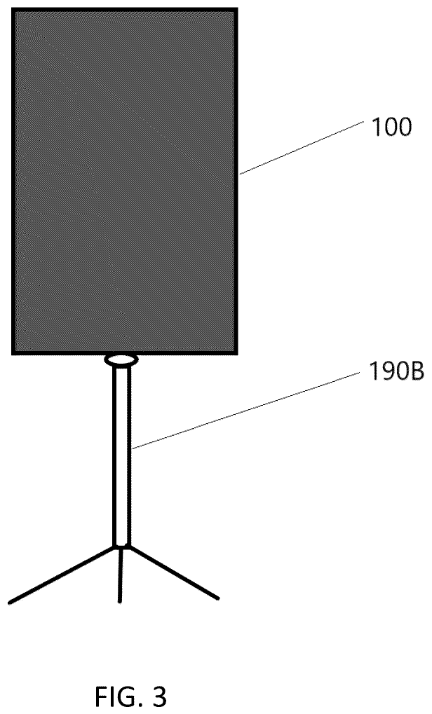

As shown in FIG. 3, the medical robot ontology 100 of an example of the present invention can include a bottom support 190B at a lower end configured to stand the medical robot body 100 on the ground thus liberating a user hand.

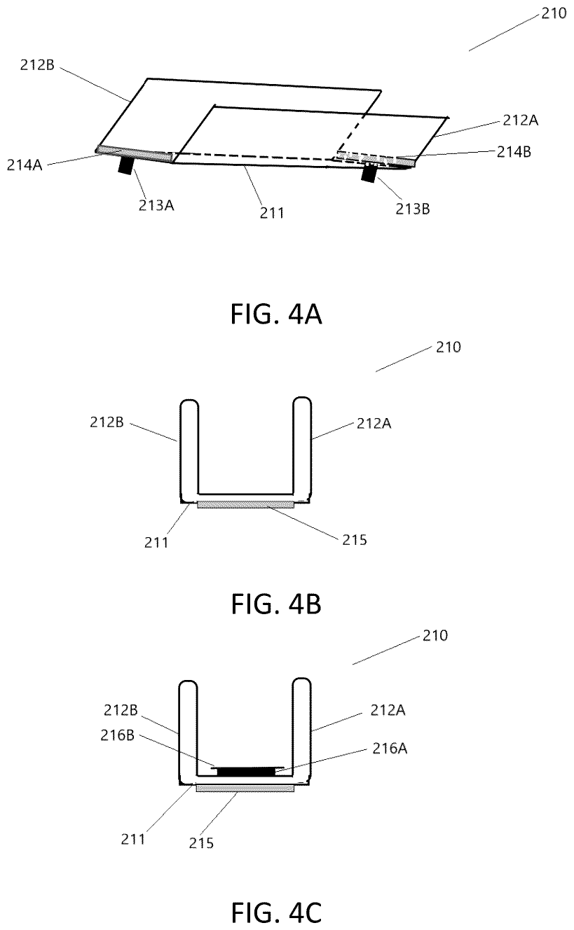

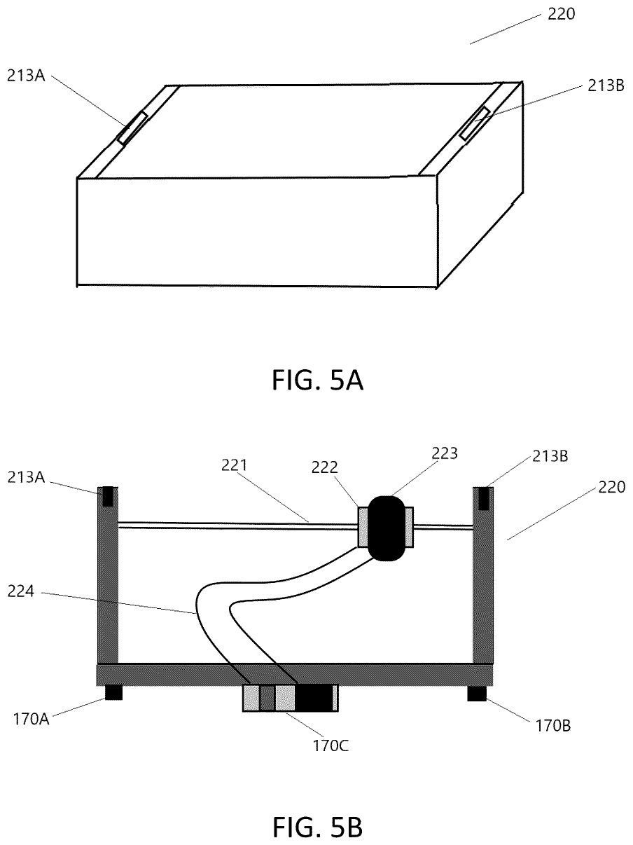

Referring to FIGS. 4 and 5, a test strip detection device 200 of a handheld medical robotic workstation of an embodiment of the present invention can comprise a groove part 210 and a probing part 220.

The groove part 210 can be a rectangular semi open structure in appearance which can include a bottom 211, a front side 212A, a back side 212B, a second left side mouthpiece 213A, a second right side mouthpiece 213B, a left side bar 214A and a right side bar 214B. The bottom 211 can pluggable connect to the probing part 220 through the second left side mouthpiece 213A and the second right side mouthpiece 213B. The left side bar 214A and the right side bar 214B can be with a height no more than 1 mm configured to prevent a test strip 216 infiltrated liquid outflow out of the groove part 210 from environment pollution. The groove part 210 can be made of transparent material, and a surface of the bottom 211 can be covered with a permeabilizing membrane 215, which can facilitate a computer vision sensor probe of the probing part 220 scanning the test strip 216.

When detecting, a reagent block side 216A of the test strip 216 is placed snugly against the bottom 211 of the grooved part 210, a substrate side 216B of the test strip 216 up, a probe head 223 of the probed part 220 can identify graphic codes of the test strip 216 and reagent blocks 216A and collect color reaction data of the reagent blocks 216A through the bottom 211 of the groove part 210 and the permeabilizing membrane 215.

As shown in FIG. 5, a detection part 220 of an embodiment of the present invention can be an upper open rectangular structure, an upper side of which can be connected to a groove part 210 through a second left side mouthpiece 213A and a second right side mouthpiece 213B, and a lower side of which can be connected to a medical robot body 100 through a first left pluggable port 170A, a first right pluggable port 170B and a plug 170C.

The detection part 220 can internal setup a detection head 223 and a lead screw system 221, a power line and a data line 224 of the detection head 223 and the lead screw system 221 can communicate with the plug 170C built-in power and data interface. The detection head 223 can be fixed to a slider 222 of the lead screw system 221. When detecting, the lead screw system 221 initiates, the detection head 223 can reciprocate with the slider 222, an illumination, an incident light emitter, a camera, a grating and a computer vision sensor on the detection head 223 begin to operate, identify and collect graphic code data of the test strip 216 and reagent block 216A, collect chromogen reaction data of reagent block 216A of the test strip 216, store and process the data, and obtain a test result. Among them, the graphic code can include a barcode, a two-dimensional code, a three-dimensional code, and a chip, and the grating can include a single grating and a multiple grating. According to the graphic code of the test strip 216 and the reagent block 216A of the test strip 216, the grating can start or close under an intelligent control, so that a best detection effect can be obtained.

The computer vision sensor of the detection part 220 can integrate a visible light, an infrared light, an ultraviolet light, a fluorescence, a laser and other multi spectra, cooperate with the illumination, incident light emitter, camera and grating, implement a detection of the test strip 216A including a variety of chemical, biological, immunological, gene chip and other reactive principle.

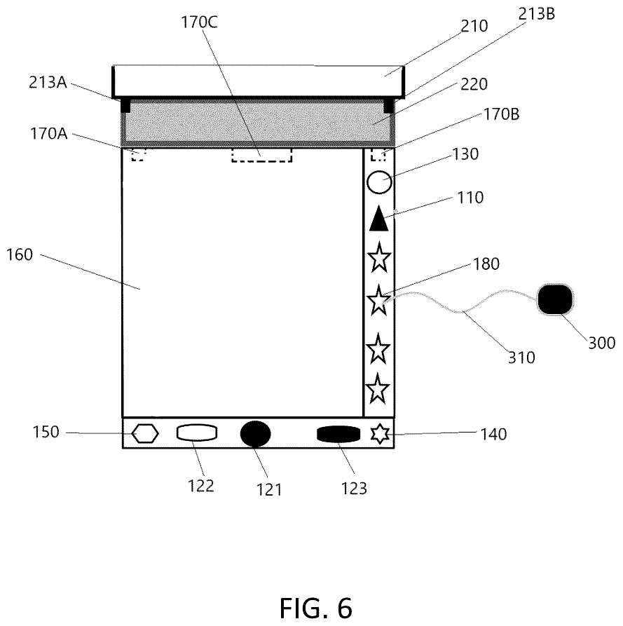

Referring to FIG. 6, which shows a combination of a handheld medical robotic workstation of an embodiment of the present invention. A medical robot body 100 as an architectural foundation, an upper end of the medical robot ontology 100 can connect to a detection part 220 through a first left pluggable port 170A, a first right pluggable port 170B and a plug 170C, and the detection part 220 can connect to the groove part 210 through a second left mouthpiece 213A and a second right mouthpiece 213B, so that a test strip 216 can implemented. The upper end of the medical robot body 100 can connect to an inspection handpiece 300 through a pluggable interface 180 and a cable line 310, so that a function of the inspection handpiece 300 can be implemented. Furthermore, a plurality of inspection handpieces 300 can match a plurality of pluggable interfaces 180 or a shared pluggable interface 180 on the upper end of the medical robot body 100, so that the handheld medical robotic workstation can implement more than one inspection.

The inspection handpiece 300 can includes, but is not limited to, a vital sign monitoring equipment, an intelligent stethoscope, an intelligent pulse detector, a computer vision sensor, an intelligent ultrasound probe, an intelligent ECG electrode lead probe, an intelligent EEG electrode lead probe, an intelligent respiratory function examination probe, an exhalation component detection sensor, an intelligent capsule gastroenteroscope, and an intelligent endoscope configured to complete a need for routine auxiliary examination. For example, the vital sign monitoring equipment can be configured to monitor temperature, pulse, respiration, blood pressure, and oxygen saturation, the intelligent stethoscope can be configured for heart and lung auscultation, the intelligent pulse detector and computer vision sensor probe can be configured to assist Chinese medicine visualization, the intelligent ultrasound probe, the intelligent ECG electrode lead probe, the intelligent EEG electrode lead probe and the intelligent respiratory function examination probe can be configured for physical examination, the exhalation component detection sensor can be configured for the examination of H. pylori, alcohol and halitosis in the stomach, the intelligent capsule gastrointestinal system can be configured for an examination of gastrointestinal system, and the intelligent endoscope can be configured to detect the natural orifice of human body.

In addition, the inspection handpiece 300 of an example of the present invention can choose a wireless scheme, which can be configured with a power supply, a probe signal emission device, a signal acquisition device, and a wireless communication device. Data collected by the inspection handpiece 300 can be transferred to a data storage and processing module of the medical robot body 100 through wireless communication, so that a freedom of the inspection handpiece 300 can be increased, and an inspection operation can be simplified.

In addition, the groove part 210, the detection part 220 of an example of the present invention can be combined with the medical robot body 100 in a inter building block manner, so that the groove part 210 can be disassembled easily, and washed and sterilized conveniently.

Referring to FIGS. 7 to 9, a blood detection pen device 400 of a handheld medical robotic workstation of an example of the present invention can comprise a housing 410, a data power cable 450, a connecting plug 460, the housing 410 can include a front hole 411, a side hole 412, a limit plate 414, and a slide slot 413, an inside of the housing 410 can set a puncture component, a blood collection component, a blood counting and classifying component, a blood electrolyte detecting component, and a waste fluid collecting sac 440. The puncture component can include a triplicate pin 421, a slide shaft 425, a spring 424, a stop slip 422, a button 423, and a slide slot 413, the slide shaft 425 can be within a turn of the spring 424, the triplicate pin 421 can be mounted at a front end of the slide shaft 425, the stop slip 422 can be mounted at the front part of the slide shaft 425, a back end of the slide shaft 425 can be free, the button 423 can be connected to a front end of the spring 424, the button 423 can be moved along the slide slot 413 on a surface of the housing 410, a back end of the spring 424 can be fixed to the housing 410 by a column 426, a front and back segments of the triplicate pin 421 can be set the stop slip 422, the triplicate pin 421 can be stretched out the housing 410 through the front hole 411, and a surface of the triplicate pin 421 can be anticoagulant coated. The blood collection component can include a capillary tube 431, a pooling tube 438 with anticoagulant coated, a first smart micropump 432 and a second smart micropump 433, a front end of the capillary tube 431 can be stretched out the housing 410 from the front hole 412. A drip of blood can flow through the capillary tube 431, the pooling tube 438, a plurality of branches of the capillary tube 431 into the blood counting and classifying component and the blood electrolyte detecting component respectively, and then driven by the first smart micropump 432 and the second smart micropump 433 into the waste collection sac 440. The blood counting and classifying component can include a miniature blood count dish 434, and a photoelectric blood count sensor 436. The blood electrolyte detecting component can include a miniature sample cell 435, and an electrochemical sensor array 437, and the electrochemical sensor array 437 can include, but is not limited to, an electrode for detecting sodium, potassium, chloride, calcium, and bicarbonate ion.

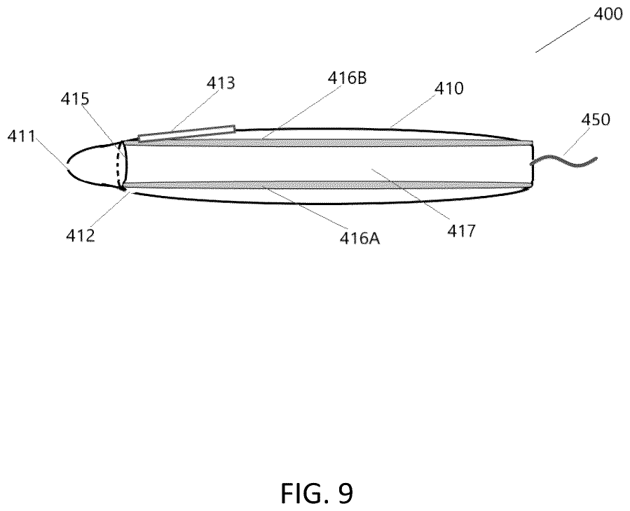

In addition, as shown in FIG. 9, the housing 410 of an embodiment of the present invention can include a detachable structure 415, which can be located at a front end of the housing 410. When the detachable structure 415 being removed from the housing 410, the puncture component can be exposed so as to install and replace the triplicate pin 421. The housing 410 can include an upper side slideway 416B, a lower side slideway 416A, and a sliding shell 417, and the sliding shell 417 can be taken down from the housing 410 along a longitudinal axis, thereby exposing the puncture component, the blood collection component, the blood cell counting and classifying component, the blood electrolyte detecting component, and the waste liquid collection sac 440, facilitating component replacement and maintenance.

During a blood collection, remove the detachable structure 415 from the housing 410, and install the triplicate pin 421, then push the sliding shaft 425 and the spring 424 backward along the slide slot 413 to the end of the chute 413 with the button 423, then press the button 423, and then the triplicate pin 421 can quickly move forward along the sliding shaft 425, and pierce the skin through the front hole 411, the blood sample can flow out continuously.

With the help of human-machine interaction, when only the blood cell analysis is detected, the first smart micropump 432 can be started, the blood sample enters a collection pipe 438 through the capillary pipe 431, and then enters the miniature blood count dish 434. The photoelectric blood count sensor 436 can conduct the blood cell classification and analysis, and obtain a blood cell classification data. Then, the cleaning solution can be inhaled through the capillary pipe 431, washed and disinfected, the blood and flushing solution enter the waste liquid collection sac 440, and then, the detachable structure 415 can be taken down from the housing 410, the triplicate pin 421 can be removed. Finally, the sliding shell 417 can be taken down from the housing 410 along the vertical axis of the upper slideway 416B and the lower slideway 416A. The puncture component, the blood collection component, the blood cell counting and classifying component, the blood electrolyte detecting component, and the waste liquid collection sac 440 can be exposed, and the waste liquid collection sac 440 can be replaced, and then insert the upper slide way 416B and the lower slide way 416A of the sliding shell 417 along the longitudinal axis into the housing 410.

With the help of human-machine interaction, when only the blood electrolyte is detected, the second smart micropump 433 can be started, the blood sample enters the collection pipe 438 through the capillary pipe 431, and enters the miniature sample cell 435, and then the electrolyte data can be detected through the electrochemical sensor array 437. Other processes can be the same as above mentioned.

With the help of human-machine interaction, when the blood cell analysis and blood electrolyte are detected at the same time, the first smart micropump 432 and the second smart micropump 433 can be started, and the blood cell analysis and blood electrolyte detection data can be obtained.

In addition, a lithium heparin can be preferred as an anticoagulant coating for the anticoagulant of the triplicate pin 421, the capillary tube 431 and the collection pipe 438. If sodium heparin, potassium heparin or ammonium heparin are selected, the detection of sodium, potassium and ammonium ions in the blood will be affected. At this time, the interference factors of sodium, potassium and ammonium ions in the anticoagulant components need to be corrected.

An environmental monitoring module of an embodiment of the present invention can include a variety of environmental monitoring sensor arrays. The monitoring items can include but are not limited to environmental temperature, humidity, light brightness, noise, CO, CO2, PM2.5, PM10, NH3, formaldehyde, and gamma rays, which can monitor a user home or working environment in real time. Moreover, a connected smart home and smart home appliances can be started or closed timely according to the user needs, thus providing the user with a good living and working environment.

A diet health monitoring module of an embodiment of the present invention can include a dry chemical detection strip, an electrochemical sensor, and a food heat and diet monitoring service software. The dry chemical detection strip and the electrochemical sensor can be configured to detect food additives, toxic and harmful substances, bacteria and toxins, and animal epidemics in diet. The food heat and diet monitoring service software can be configured to identify food categories, estimate heat values and nutrient content, help family members develop healthy eating habits.

A weight/exercise management module of an embodiment of the present invention can include a weight meter, a measuring ruler, an intelligent body fat detector, and an intelligent wearable device. Body mass index (BMI) can be obtained from weight data and height data, and skeletal muscle content, water ratio and fat content of different parts can be obtained from intelligent body fat detector to assist family members to obtain a personalized diet, an exercise program recommendations and planning.

Referring to FIGS. 6, 7 and 10, a component storage box 500 of the handheld medical robot workstation of an embodiment of the present invention can include a handle 510, a cover 520, a lock 530, a box body 550, a clamp 540, a power adapter 570, a power socket 580, and a power cord 560. The box body 550 can include a battery 551 and a plurality of storage bins 552, and the storage bin 552 can include a circuit interface 553 configured to store components. The size and shape of the storage cell 552 can be compatible with the storage components. The collected components can include but are not limited to the groove part 210, the detection part 220, the inspection handpiece 300, and the blood detection pen device 400. The box body 550 can include a storage tank 554 without circuit interface configured to store reagent consumables. The components can be charged after being put into the box body 550, and can be installed and combined with the medical robot body 100 after being taken out to perform diagnosis and treatment.

A plurality of reagent consumables 600 of an embodiment of the present invention can include a dry chemical method test strip 216, a colloidal gold test strip 216, a buffer solution, a cleaning solution, a triplicate pin 421, a capillary tube 431, and a waste liquid collection sac 440. The dry chemical method test strip 216 and the colloidal gold test strip 216 can be provided with a graphic code and a reagent block 216A. The graphic code can be a bar code, a two-dimensional code or chip, which can include a basic information of the test strip 216, such as a type of the test strip 216, a plurality of test items arranged in order, a detection spectral wavelength, a positive quantitative standard and a negative standard, a date of delivery, an expiry date, etc. Items of the dry chemical test strip 216 can include but are not limited to a urine routine, a stool routine, a saliva routine, a leucorrhea routine, a prostate fluid routine, a blood glucose, a blood lipid, a blood creatinine and urea nitrogen, a blood transaminase, a blood myocardial enzyme, and toxic and harmful ingredients in food.

An application method 10000 of the handheld medical robot workstation of an embodiment of the present invention can comprise a plurality of steps as follows.
11000: A user submits an application for acquiring the handheld medical robot workstation.
12000: The user accepts simulation training to be familiar with a basic structure, assembly and usage of the handheld medical robot workstation in a remote expert service manner.
13000: The user deploys the handheld medical robot workstation, determines health and medical needs of family members, selectively configures device combination and service scheme of the handheld medical robot workstation.

The medical robot body can be a basic configuration, which can be added with a test strip detection device, an environmental monitoring device, a plurality of inspection handpieces, a blood detection pen device, a data service, and reagent consumables. The handheld medical robot workstation can adopt modular configuration and plug-and-pull block assembly, set up family member biometrics in human-machine interaction mode, and establish a one-to-one corresponding connection with the family members' smart wearable device and smart mobile terminal, and create the family members' medical health files, and apply for data services, which can include data storage, data transmission, data encryption, data calculation and data mining.
14000: The user receives daily health management services, which can include a home environment monitoring, a diet health monitoring, a weight/exercise management, and a vital signs monitoring.
15000: The user receives a physical examination service at home.
16000: The user receives a medical inquiry service, which can include an appointment, an examination, a diagnosis, a treatment and follow-up.
17000: A server receives family members' health diagnosis and treatment data, and updates health medical records of family members in real time.

A home environment monitoring method 14100 of the handheld medical robot workstation of an embodiment of the present invention can include a plurality of steps as follows.
14110: The handheld medical robot workstation can be equipped with an environmental monitoring module and a plurality of sensors. The environmental monitoring items can include temperature, humidity, light brightness, noise, CO, CO2, PM2.5, PM10, NH3, formaldehyde and gamma rays.
14120: A user starts the handheld medical robot workstation.
14130: The user selects an environmental monitoring option, and starts the environmental monitoring, and obtains monitoring data on a real-time dynamic interface.
14140: The user sets a plurality of household environmental health indicator ranges, and if the monitoring data exceed the household environmental health indicator ranges, the handheld medical robot workstation can start a smart home, smart wear, and smart service robots being interconnected, and implement an opening and closing of doors and windows, curtains, air conditioning, air purifier, and sweeping robot wiping the floor.
14150: In case of extreme household environmental health indicator value, the handheld medical robot workstation can send a warning and send it to a mobile smart terminal of family members in time. If necessary, the handheld medical robot workstation can start a fire extinguishing device and send out a fire alarm help information.

A diet health monitoring method 14200 of the handheld medical robot workstation of an embodiment of the present invention can include a plurality of steps as follows.
14210: The handheld medical robot workstation can be equipped with a diet monitoring module, a plurality of sensors and service software. The diet monitoring module can include a dry chemical test strip, an electrochemical sensor, a food calories and diet monitoring service software.
14220: The handheld medical robot workstation detects food safety, which includes the dry chemical test strip and electrochemical sensors to detect food additives, toxic and harmful substances, bacteria and toxins, and animal epidemics in the diet.
14230: The handheld medical robot workstation detects a caloric value and composition of the diet, which including taking pictures of the food, fruit and drink eaten by family members every day, detecting its sugar and salt concentrations, and identifies the food category, estimates the caloric value and the content of six major nutrients including protein, fat, sugar, vitamins, minerals, and cellulose, so as to obtain a total caloric value and nutritional composition of daily diet of family members.
14240: The handheld medical robot workstation tracks a dietary habit and dietary behavior characteristic of family members, and combines home-based physical examination indicators to comprehensively evaluate a dietary health of family members, so as to submit personalized healthy dietary recommendations and reference target values for family members. The daily dietary record data of family members can be compared with the reference target values, so as to remind family members of reasonable and balanced diet at all times, assist each family member to gradually develop their own healthy eating habits.

A weight/motion management method 14300 of the handheld medical robot workstation of an embodiment of the present invention can include a plurality of steps as follows.
14310: The handheld medical robot workstation can be equipped with a weight management module, which can include a weight meter, a measuring ruler, an intelligent body fat detector, and an intelligent wearable device.
14320: A user inputs a measured weight data and height data into the workstation through human-machine interaction to obtain the BMI of each family member. If the BMI of family members is too large, the handheld medical robot workstation can suggest family members a necessary to appropriately reduce food intake and increase exercise, and if the BMI of family members is too small, the handheld medical robot workstation can indicate family members a need for proper nutrition.
14330: the intelligent body fat detector obtains a skeletal muscle content, a water ratio, and a fat content of different human body parts, and assists family members to obtain personalized diet, exercise program recommendations and planning.

A vital sign monitoring method 14400 of the handheld medical robot workstation of an embodiment of the present invention can include a plurality of steps as follows.
14410: The handheld medical robot workstation can be equipped with a vital sign monitoring equipment, which including an intelligent body surface temperature sensor probe, an intelligent body surface pulse detection sensor probe, an intelligent body surface respiration detection sensor probe, an intelligent blood pressure detection pressure sensor cuff, and an intelligent body surface oxygen saturation detection sensor probe.
14420: A user starts the handheld medical robot workstation.
14430: The user selects the vital sign monitoring item in a touch screen display, selects one or more monitoring options of body temperature, pulse, respiration, blood pressure and blood oxygen saturation, takes out the intelligent detection sensor probe, and starts a monitoring by referring to a demonstration guide of the touch screen display, then the touch screen display can display real-time dynamic monitoring data.
14440: The user sets a vital signs and health indicators value range for family members. If the vital signs and health indicators value are beyond the vital signs and health indicators value rang, especially in an extreme case, the touch screen display can send a warning, and the vital signs and health indicators value can be sent to a contracted family doctor or Internet hospital synchronously. If necessary, a medical emergency information can be sent.

A home physical examination method 15000 of the handheld medical robot workstation of an embodiment of the present invention can include a plurality of steps as follows.
15100: According to a home health plan, family members plan times and date of annual physical examination.
15200: According to a recommendation of a contracted family doctor or Internet hospital, family members can set up personalized physical examination items.
15300: A user rents one or more inspection handpieces and blood detection pen device from a service provider, and purchase required reagent consumables.
15400: Under a remote guidance of the contracted family doctor or Internet hospital, a home-based self-help physical examination can be carried out. The physical examination items can be carried out according to an electronic form and human-machine interaction prompt, which including a general physical examination, a routine surgery, an internal medicine, a gynecology, a pediatrics, a dermatology, an otorhinolaryngology, a blood routine, a urine routine, a stool routine examination, a blood biochemical examination, a visceral body surface mass breast ultrasound, an ECG, an EEG, a respiratory function examination, a Helicobacter pylori in the stomach, a video data of motion joint activity, a facial image, a tongue image and pulse image of traditional Chinese medicine.
15500: The medical examination data of family members can be encrypted and published to the health files of family members on a cloud server.
15600: The contracted family doctor and Internet hospital can obtain the medical examination data of family members, and give a medical and health advice, if necessary, arrange for family members to see a doctor to obtain further diagnosis and treatment services.

A user seeking medical advice method 16000 of the handheld medical robot workstation of an embodiment of the present invention can include a plurality of steps as follows.
16100: If a family member feels unwell, a user can make an appointment with a contracted family doctor or Internet smart hospital.
16200: The family member can implement a detection of body temperature, pulse, respiration, blood pressure, blood oxygen saturation with a help of a vital sign monitoring equipment, and traditional Chinese medicine and physical examination with a help of an intelligent stethoscope, an intelligent pulse detector, and a computer vision sensor probe in a human-machine interaction mode and remote consultation.
16300: The family member can implement an auxiliary examination, including an excreta detection, a blood detection and a handpiece inspection.
16400: The family member can obtain a diagnosis. The contracted family doctor or Internet hospital makes a clear diagnosis based on a main complaint, a past medical history, a current medical history, a physical examination and an auxiliary examination data of the family member. If necessary, according to a recommendation of the family doctor or Internet hospital an additional physical examination and auxiliary examination can be performed to further clarify the diagnosis.
16500: The family member can obtain a diagnosis and treatment plan. The contract family doctor or the Internet hospital can invite family members to participate in, jointly develop a personalized diagnosis and treatment plan, issue a prescription, and send the prescription to a drug and device manufacturer.
16600: If the diagnosis includes critical illness, trauma and other situations requiring emergency treatment, the handheld medical robot workstation can start an emergency mechanism, arrange for a nearest hospital to go for rescue, or the family member go to the nearest hospital to receive treatment.
16700: The family member obtains a treatment. The family member can adopt health advice, receive drugs and treatment devices sent by logistics, and enter a treatment process.
16800: The family member obtains a regular follow-up service.

An excreta detection method 16310 of the handheld medical robot workstation of an embodiment of the present invention can include a plurality of steps as follows.
16311: A user prepares one or more test strips for the excreta detection according to a suggestion of a contracted family doctor or Internet hospital.
16312: The user connects the medical robot body with a groove component and a detection component.
16313: The user collects excreta samples of family member, such as urine, feces and vomitus.
16314: The user soaks and absorbs a plurality of reagent blocks of the test strips with excreta samples.
16315: The user puts the test strips into the groove component with the reagent blocks facing a bottom of the groove component.
16316: The user starts the handheld medical robot workstation to detection.
16317: A detector head of the detection component moves back and forth with a slider of a lead screw system to identify an information of the test strip and reagent blocks.
16318: When an end point of reagent blocks reaction time is reached, the detector head scans the reagent blocks, collects data, processes data, and obtains a detection result. The end point of reagent block reaction time can be corrected by the reaction end point time under a standard conditions of reagent block combined with an ambient temperature, humidity, and altitude.

A blood detection method 16320 of the handheld medical robot workstation of an embodiment of the present invention can include a plurality of steps as follows.
16321: A user punctures a family member with a blood collection component of a blood detection pen device at the end of a finger abdomen, earlobe or toe, and then blood can flow out automatically.
16322: A first smart micropump and a second smart micropump of the blood detection pen device are started, and the blood enters a capillary tube and the collection pipe.
16323: A part of the blood enters a blood cell counting and classifying component, enters a miniature blood cell count dish, and a photoelectric blood count sensor carries out the blood cell classification analysis to obtain the blood cell classification data.
16324: A part of blood enters the collection pipe through the capillary tube, enters a miniature sample cell, and obtains data of sodium ion, potassium ion, chloride ion, calcium ion and bicarbonate of blood samples through an electrochemical sensor array.
16325: According to a need, a blood lipid dry chemical test strip can absorb the blood, when reaching an end point of a reaction, the blood lipid dry chemical test strip can be placed into the detection component to obtain a quantitative data of blood total cholesterol, high-density lipoprotein cholesterol, triglyceride, and low-density lipoprotein cholesterol.
16326: According to a need, a glutamic-pyruvic transaminase (GPT) dry chemical test strip can absorb the blood, when reaching an end point of a reaction, the GPT dry chemical test strip can be placed into the detection component to obtain a quantitative data of blood GPT and evaluate a liver function.
16327: According to a need, a creatinine and urea nitrogen dry chemical test strip can absorb the blood, when reaching an end point of a reaction, the creatinine and urea nitrogen dry chemical test strip test strip can be placed into the detection component detection device to obtain a quantitative data of blood creatinine and urea nitrogen, and evaluate a renal function.
16328: According to a need, a myocardial zymogram dry chemical test strip can absorb the blood, when reaching an end point of a reaction, the myocardial zymogram dry chemical test strip can be placed into the detection component detection device to obtain a quantitative data of blood myocardial zymogram and evaluate a degree of myocardial injury.
16329: According to a need, a blood glucose dry chemical test strip can absorb the blood, when reaching an end point of a reaction, the blood glucose dry chemical test strip can be placed into the detection component detection device to obtain a quantitative data of blood glucose and evaluate an insulin secretion function of the pancreas.

An inspection handpiece method 16330 of the handheld medical robot workstation of an embodiment of the present invention can include a plurality of steps as follows.
16331: A user prepares the inspection handpieces according to an advice of a contracted family doctor or Internet smart hospital.
16332: According to a need, the user takes out an ultrasonic probe inspection handpiece from a component storage box, connects the ultrasonic probe inspection handpiece to an interface of the medical robot body, selects the ultrasonic inspection item, selects a body part, starts an ultrasonic detection with a help of human-machine interaction, detects organs, body surface lumps, etc., and obtains the ultrasonic inspection data.
16333: According to a need, the user takes out the ECG, EEG and respiratory function inspection handpieces from the component storage box, connects the ECG, EEG and respiratory function inspection handpieces to interfaces of the medical robot body, selects a corresponding inspection item, starts the corresponding inspection, implements the inspection in a human-machine interaction mode, and obtains the ECG, EEG, respiratory function and expiratory component inspection data.
16334: According to a need, the user takes out an endoscope from the component storage box, enters through the mouth, nasal cavity, external ear canal, vagina and anus, and connects the medical robot body to obtain an inspection data of family member oral pharynx, nasal cavity, external ear canal, vagina, anus and rectum.
16335: According to a need, the user takes out an intelligent capsule gastroscope from the component storage box, a family member swallows the intelligent capsule gastroscope through mouth, and connects the medical robot body to obtain a gastrointestinal inspection data.

A handheld medical robot workstation system of an embodiment of the present invention can includes a 5G, a medical AI, an Internet of Things system, a supply chain system, a logistics system, a cloud service, an operating system, and an application software, which can execute an instructions issued by a data storage and processing module to assist in completing an operation of the handheld medical robot workstation.

A computer storage medium of an embodiment of the present invention can store a computer program, which can be used by a processor to execute a handheld medical robot workstation, a third-party application system and its application workflow.

The present invention is intended to cover any variant, use or adaptive change of the present invention, which follows the general principles of the present invention and includes the common knowledge or commonly used technical means in the technical field not disclosed by the present invention. The description and embodiments are only considered as examples, and the scope of the invention is limited only by the appended claims.

The above mentioned is only the embodiment of the invention and does not limit the technical scope of the invention. Therefore, any minor modifications, equivalent changes and modifications made to the above embodiments according to the technical essence of the invention still fall within the scope of the technical solution of the invention. Professionals should be aware that they can use different methods to achieve the described functions for each specific application, but such implementation should not be considered beyond the scope of the invention.

### INDUSTRIAL APPLICABILITY

(1) Practicability: Ordinary technical personnel in the technical field can objectively reproduce the subject matter of the invention after reading the overall technical content disclosed in the application document.

(2) Reproducibility: According to the disclosed technical content, ordinary technical personnel in the technical field can repeatedly implement the technical solution adopted in this application to achieve its purpose. Such repeated implementation has no quantitative limit, does not rely on any random factors, and the results of each implementation are the same.

(3) Beneficiality: The handheld intelligent medical workstation in the technical solution of this application can produce positive economic and social benefits.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

1-15. (canceled)

16: A handheld medical robotic workstation, implementing health care operations self-help at home, comprising:
a medical robotic body configured to support the handheld medical robotic workstation operation;
a test strip detection device configured to scan a test strip;
a plurality of inspection handpieces configured to implement a physical examination; and
a blood detection pen device configured to collect and assay a blood sample.

17: The handheld medical robotic workstation according to claim 16, wherein the medical robotic body comprises a pluggable slot module, the pluggable slot module includes a circuit interface, a data interface, and a bayonet, configured to connect the test strip detection device, the inspection handpieces and the blood detection pen device to the medical robotic body electrically in a pluggable manner.

18: The handheld medical robotic workstation according to claim 17, wherein the medical robotic body further comprises a power supply module, a wireless communication module, a data storage and processing module, a display interface module, an environmental monitoring module, an operation button module, a biometric module, a human-machine interaction module and a data security module.

19: The handheld medical robotic workstation according to claim 18, wherein the medical robotic body further comprises:
a dorsal bracket configured to stand obliquely on a desktop, and
a bottom bracket configured to stand upright on a ground.

20: The handheld medical robotic workstation according to claim 16, wherein the test strip detection device comprises:
a groove part configured to place a test strip, and
a detection part configured to scan the test strip, the detection part connects with the groove part pluggable and with the medical robotic body electrically pluggable.

21: The handheld medical robotic workstation according to claim 20, wherein the detection part comprises a detection head and a lead screw system, the detection head is fixed on a slider of the lead screw system configured to move with the slider for scanning the test strip.

22: The handheld medical robotic workstation according to claim 1, wherein the inspection handpiece comprises:
a vital sign detection apparatus configured to monitor vital signs, and
a physical examination apparatus configured to implement physical examination.

23: The handheld medical robotic workstation according to claim 22, wherein the physical examination apparatus comprises an intelligent stethoscope, an intelligent pulse image detector, a computer vision sensor probe, an intelligent ultrasound probe, an intelligent electrocardiogram electrode lead probe, an intelligent electroencephalogram electrode lead probe, an intelligent respiratory function examination probe, an exhalation component detection sensor, an intelligent capsule gastrointestinal endoscope, and an intelligent endoscope.

24: The handheld medical robotic workstation according to claim 22, wherein the inspection handpiece further comprises a power supply, a wireless communication component, a signal emission component and a signal acquisition component configured to collect data and send data to a medical robot body via wireless communication.

25: The handheld medical robotic workstation according to claim 16, wherein the blood detection pen device comprises:
a puncture component configured to pierce a skin,
a blood collection component configured to collect a blood sample,
a blood cell counting and classifying component configured to analyze the blood sample cell composition,
a blood electrolyte detecting component configured to analyze the blood sample plasma components,
a waste liquid collecting sac configured to pool a waste, and
a housing configured to accommodate the puncture component, the blood collection component, the blood cell counting and classifying component, the blood electrolyte detecting component and the waste liquid collecting sac.

26: The handheld medical robotic workstation according to claim 25, wherein the blood collection component comprises a capillary tube configured to communicate the blood cell counting and classifying component and/or the blood electrolyte detecting component, a smart micropump, and the waste liquid collecting sac.

27: The handheld medical robotic workstation according to claim 16, further comprising a plurality of reagent consumables which includes a test strip, a buffer, a cleaning detergent, a triangular needle, a capillary tube, and a waste collection sac.

28: The handheld medical robotic workstation according to claim 27, further comprising a component storage box configured to temporarily store a test strip detection device, a plurality of inspection handpieces, a blood detection pen device, and a plurality of reagent consumables.

29: The handheld medical robotic workstation according to claim 28, wherein the component storage box comprises a power adapter, a power socket, a power cord, a battery, and a plurality of storage cells.

30: A method of a handheld medical robotic workstation, comprising:
deploying the handheld medical robot workstation;
implementing a daily health management, which including a home environment monitoring, a diet health monitoring, a weight management, and a vital sign monitoring;
carrying out a health examination at home;
seeking a medical service; and
creating a family member health file.

31: The method according to claim 30, wherein the deploying the handheld medical robotic workstation comprises:
configuring the handheld medical robotic workstation,
assembling a medical robotic body and a plurality of functional modules, and
creating a medical and health data file for the family member.

32: The method according to claim 30, wherein the vital sign monitoring comprises:
equipping the handheld medical robotic workstation with a vital sign function module,
selecting the vital sign monitoring interface, and obtaining the vital sign data, and
sending the vital sign data to a family doctor.

33: The method according to claim 30, wherein the health examination at home comprises:
planning the family member physical examination,
personalizing a plurality of physical examination items for the family member,
renting a plurality of inspection handpieces and a blood detection pen device, and purchasing a plurality of reagent consumables,
implementing self-service physical examination at home,
encrypting the medical examination data and uploading to the family member health file on a server, and
obtaining a medical and health advice from a family doctor.

34: The method according to claim 30, wherein seeking a medical service comprises:
making an appointment with a family doctor,
monitoring vital signs and implementing physical examination self-service for the family member,
making a diagnosis and treatment plan, and
implementing a treatment.

35: A system of a handheld medical robotic workstation, comprising a handheld medical robotic workstation, a medical AI, an Internet of Things system, a supply chain system, a logistics system, a cloud service, an operating system, and an application software configured to implement a self-service health diagnosis and treatment at home.
